(19) United States
(12) Patent Application Publication
(10) Pub. No.: US 2026/0192108 A1
(43) Pub. Date: Jul. 9, 2026

(54) Implantable Stimulator Device With Tissue Biasing for MRI Protection

(71) Applicant: Boston Scientific Neuromodulation Corporation, Valencia, CA (US)

(72) Inventors: Kiran K. Gururaj, Santa Clarita, CA (US); David Wagenbach, Simi Valley, CA (US); Goran N. Marnfeldt, Valencia, CA (US)

(21) Appl. No.: 19/429,807

(22) Filed: Dec. 22, 2025

Related U.S. Application Data
(60) Provisional application No. 63/742,766, filed on Jan. 7, 2025.

(51) Int. Cl. A61N 1/08; A61N 1/36

(52) U.S. Cl. CPC A61N 1/08; A61N 1/36125; A61N 1/36153; A61N 1/36157

(57) ABSTRACT

Stimulator circuitry useable in a stimulator device is described. The stimulator circuitry features an MRI-safe mode configured to protect the device and the patient from malfunctions caused by voltages that may develop because of electromagnetic fields and gradients present in a patient’s tissue during an MRI procedure. The circuitry is configured to assert a common mode voltage to the patient’s tissue that is preferably between a ground and a compliance voltage of the stimulator device.

### CROSS REFERENCE TO RELATED CASES

This is a non-provisional application of U.S. Provisional Patent Application Serial No. 63/742,766, filed January 7, 2025, which is incorporated herein by reference in its entirety, and to which priority is hereby claimed.

### FIELD OF THE INVENTION

This application relates to Implantable Medical Devices (IMDs), and more specifically to implantable stimulator devices having MRI protection.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) or Deep Brain Stimulation (DBS) system. However, the present invention may find applicability with any stimulator device system.

### SUMMARY

Disclosed herein is an implantable stimulator device configured to provide electrical stimulation to a patient, comprising: a plurality of electrode nodes each couplable to a different electrode configured to contact the patient’s tissue, stimulation circuitry configured to drive at least two of the electrode nodes to provide current through the tissue, biasing circuitry configured to produce a reference voltage, a plurality of first switches each associated with a different one of the electrode nodes, wherein each of the plurality of first switches is configured, when selected, to couple the reference voltage to its associated electrode node to provide a common mode voltage to the patient’s tissue at that node’s associated electrode, and control circuitry configured to: receive a command to put the stimulator device in an MRI-safe mode, and in response to the command, closing a selected one or more of the first plurality of first switches. According to some embodiments, the selected one or more of the first plurality of switches comprises a first switch associated with a case electrode. According to some embodiments, the selected one or more of the first plurality of switches comprises a first switch associated with an electrode comprised upon an electrode lead that is implantable in the patient’s tissue. According to some embodiments, the stimulation circuitry is configured to be powered between a compliance voltage (VH) and a ground, and wherein the reference voltage is between VH and ground. According to some embodiments, the reference voltage is VH/2. According to some embodiments, the control circuitry is configured to increase VH upon receipt of the command to put the stimulator device in an MRI-safe mode. According to some embodiments, the biasing circuitry comprises a high impedance driver circuitry and a programmable driver circuitry. According to some embodiments, the high impedance driver circuitry comprises a voltage divider. According to some embodiments, the programmable driver circuitry comprises an amplifier configured to produce the reference voltage. According to some embodiments, the amplifier is configured to receive a programmable input bias current that may be programmably selected to control a drive strength of the reference voltage. According to some embodiments, the control circuitry is configured to select between the high impedance driver circuitry and the programmable driver circuitry in the MRI-safe mode. According to some embodiments, the control circuitry is configured to cause the device to cease providing stimulation when in the MRI-safe mode. According to some embodiments, the device is configured to provide stimulation at selected one or more of the electrode nodes when the device is in the MRI-safe mode. According to some embodiments, the stimulation comprises at least one active stimulation duration during which the stimulation circuitry actively drives current at at least two selected stimulation electrode nodes, and wherein the control circuitry is configured to: select the at least two stimulation electrode nodes, and select one or more biasing electrode nodes for providing the common mode voltage to the patient’s tissue. According to some embodiments, the control circuitry is configured to, in response to the command to put the stimulator device in an MRI-safe mode, close the one or more of the plurality of first switches for the biasing electrode nodes. According to some embodiments, the control circuitry is configured to cause the stimulation circuitry to drive the at least two stimulation electrode nodes during the stimulation duration. According to some embodiments, the control circuitry is configured to select between the high impedance driver circuitry and the programmable driver circuitry in response to the command to put the MRI-safe mode. According to some embodiments, the stimulation comprises at least one passive charge recovery duration during which the stimulation does not drive the at least two stimulation electrode nodes, and wherein the control circuitry is configured to close second switches coupled to the at least two stimulation electrode nodes during the passive charge recovery duration to drain charge from DC-blocking capacitors coupled to the at least two stimulation electrode nodes. According to some embodiments, the control circuitry is configured to close second switches coupled to the one or more biasing electrode nodes during the passive charge recovery duration to drain charge from DC-blocking capacitors coupled to the biasing electrode nodes. According to some embodiments, the command to put the stimulator device in an MRI-safe mode is responsive to a sensed magnetic field.

Also disclosed herein is an implantable stimulator device configured to provide electrical stimulation to a patient, comprising: a plurality of electrode nodes each couplable to a different electrode configured to contact the patient’s tissue, stimulation circuitry configured to drive at least two of the electrode nodes to provide current through the tissue, biasing circuitry configured to produce a reference voltage, wherein the biasing circuitry comprises a high impedance driver circuitry and a programmable driver circuitry, control circuitry configured to: receive a command to put the stimulator device in an MRI-safe mode, and in response to the command: select one of either the high impedance driver circuitry or the programmable driver circuitry, connect the selected driver circuitry to one or more of the a plurality of electrode nodes, and use the selected driver circuitry to provide the reference voltage to the patient’s tissue via the selected electrode nodes. According to some embodiments, the high impedance driver circuitry comprises a voltage divider. According to some embodiments, the programmable driver circuitry comprises an amplifier configured to produce the reference voltage. According to some embodiments, the amplifier is configured to receive a programmable input bias current that may be programmably selected to control a drive strength of the reference voltage.

### BRIEF DESCRIPTION OF THE DRAWINGS

FIG. 1 shows an Implantable Pulse Generator (IPG), in accordance with the prior art.

FIGS. 2A and 2B show an example of stimulation pulses producible by the IPG, in accordance with the prior art.

FIG. 3 shows stimulation circuitry useable in the IPG, in accordance with the prior art.

FIG. 4 shows various external devices capable of communicating with and programming stimulation in an IPG, in accordance with the prior art.

FIG. 5 shows an improved IPG having neural response sensing capability.

FIG. 6 shows stimulation producing a neural response, and the sensing of that neural response at least one electrode of the IPG.

FIG. 7 shows biasing circuitry useable to set a common mode voltage Vcm to the patient’s tissue.

FIG. 8 illustrates an embodiment of IPG circuitry as described herein.

FIG. 9 illustrates a resistance network for passive charge recovery.

FIG. 10 illustrates tissue voltages induced during an MRI procedure.

FIG. 11 illustrates an example of an MRI-safe mode during the provision of monopolar stimulation.

FIG. 12 illustrates an example of an MRI-safe mode during the provision of bipolar stimulation.

### DETAILED DESCRIPTION

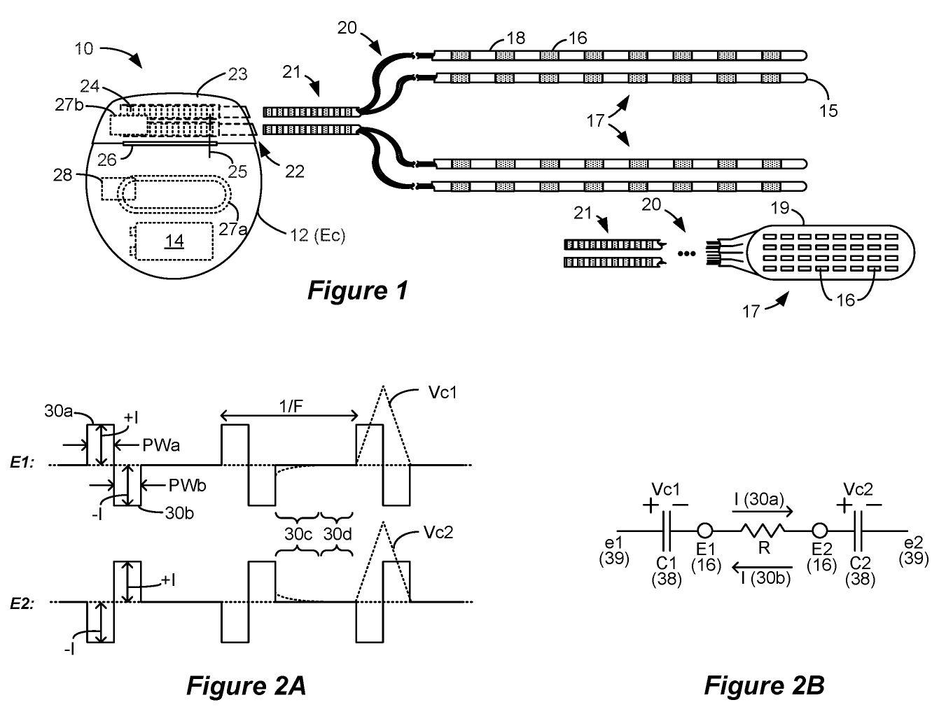

A stimulator system typically includes an Implantable Pulse Generator (IPG) 10 shown in FIG. 1. The IPG 10 includes a biocompatible device case 12 that holds the circuitry and a battery 14 for providing power for the IPG to function. The IPG 10 is coupled to tissue-stimulating electrodes 16 via one or more electrode leads that form an electrode array 17. For example, one or more percutaneous leads 15 can be used having ring-shaped or split-ring electrodes 16 carried on a flexible body 18. In another example, a paddle lead 19 provides electrodes 16 positioned on one of its generally flat surfaces. Lead wires 20 within the leads are coupled to the electrodes 16 and to proximal contacts 21 insertable into lead connectors 22 fixed in a header 23 on the IPG 10. The header can comprise an epoxy for example. Once inserted, the proximal contacts 21 connect to header contacts 24 within the lead connectors 22, which are in turn coupled by feedthrough pins 25 through a case feedthrough 26 to stimulation circuitry 28 within the case 12.

In the illustrated IPG 10, there are thirty-two electrodes (E1-E32), split between four percutaneous leads 15, or contained on a single paddle lead 19, and thus the header 23 may include a 2x2 array of eight-electrode lead connectors 22. However, the type and number of leads, and the number of electrodes, in an IPG is application specific and therefore can vary. The conductive case 12, or some conductive portion of the case, can also comprise an electrode (Ec). In an SCS application, the electrode lead(s) are typically implanted in the spinal column proximate to the dura in a patient’s spinal cord, preferably spanning left and right of the patient’s spinal column. The proximal contacts 21 are tunneled through the patient’s tissue to a distant location such as the buttocks where the IPG case 12 is implanted, at which point they are coupled to the lead connectors 22. In a DBS application, the electrode leads are implanted in the brain through holes in the skull, and lead extensions are used to connect the leads to the IPG which is typically implanted under the clavicle (collarbone). In other IPG examples designed for implantation directly at a site requiring stimulation, the IPG can be lead-less, having electrodes 16 instead appearing on the body of the IPG 10 for contacting the patient’s tissue. The IPG lead(s) can be integrated with and permanently connected to the IPG 10 in other solutions. SCS therapy can relieve symptoms such as chronic back pain, while DBS therapy can alleviate Parkinsonian symptoms such as tremor and rigidity.

IPG 10 can include an antenna 27a allowing it to communicate bi-directionally with a number of external devices discussed subsequently. Antenna 27a as shown comprises a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. In FIG. 1, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Zigbee, WiFi, MICS, and the like.

Stimulation in IPG 10 is typically provided by pulses each of which may include a number of phases (30i), as shown in the example of FIG. 2A. Stimulation parameters typically include amplitude (current I, although a voltage amplitude V can also be used); frequency (F); pulse width (PW); the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode E1 has been selected as an anode (during its first phase 30a), and thus provides pulses which source a positive current of amplitude +I to the tissue. Electrode E2 has been selected as a cathode (again during first phase 30a), and thus provides pulses which sink a corresponding negative current of amplitude -I from the tissue. This is an example of bipolar stimulation, in which the lead includes one anode pole and one cathode pole. Note that more than one electrode on the lead may be selected to act as an anode electrode to form an anode pole at a given time, and more than one electrode may be selected to act as a cathode to form a cathode pole at a given time, as explained further in USP 10,881,859. Stimulation provided by the IPG 10 can also be monopolar, although this example is not yet shown. In monopolar stimulation, the lead is programmed with a single pole of a given polarity (e.g., a cathode pole), with the conductive case electrode Ec acting as a return (e.g., an anode pole). Again, more than one electrode on the lead may be active to form the pole during monopolar stimulation.

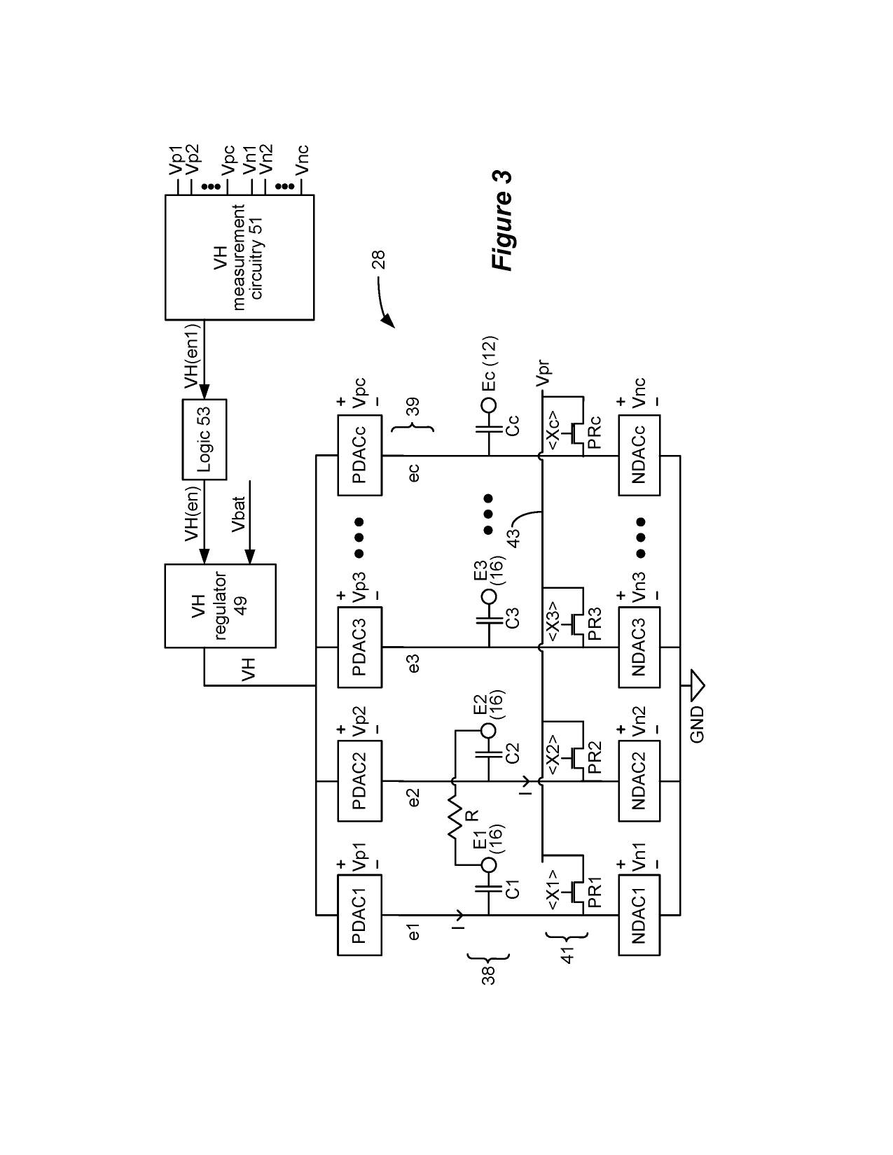

IPG 10 as mentioned includes stimulation circuitry 28 to form prescribed stimulation at a patient’s tissue. FIG. 3 shows an example of stimulation circuitry 28, which includes one or more current source circuits and one or more current sink circuits. The sources and sinks constitute driver circuitry configured to drive current through the patient’s tissue. The sources and sinks can comprise Digital-to-Analog converters (DACs), and may be referred to as PDACs and NDACs in accordance with the Positive (sourced, anodic) and Negative (sunk, cathodic) currents they respectively issue. In the example shown, a NDACi/PDACi pair is dedicated (hardwired) to a particular electrode node ei 39. Each electrode node ei 39 is connected to an electrode Ei 16 via a DC-blocking capacitor Ci 38, for the reasons explained below. The stimulation circuitry 28 in this example also supports selection of the conductive case 12 as an electrode (Ec 12), which case electrode is typically selected for monopolar stimulation as explained above. PDACs and NDACs can also comprise voltage sources.

Proper control of the PDACs and NDACs allows any of the electrodes 16 to act as anodes or cathodes to create a current through a patient’s tissue, R, hopefully with good therapeutic effect. Consistent with the example provided in FIG. 2A, FIG. 2B shows operation during the first phase 30a in which electrode E1 has been selected as an anode electrode to source current I to the tissue R and E2 has been selected as a cathode electrode to sink current from the tissue. Thus, PDAC1 and NDAC2 are digitally programmed to produce the desired current, I, with the correct timing (e.g., in accordance with the prescribed frequency and pulse widths). As mentioned above, more than one anode electrode and more than one cathode electrode may be selected at one time, and thus current can flow through the tissue R between two or more of the electrodes 16.

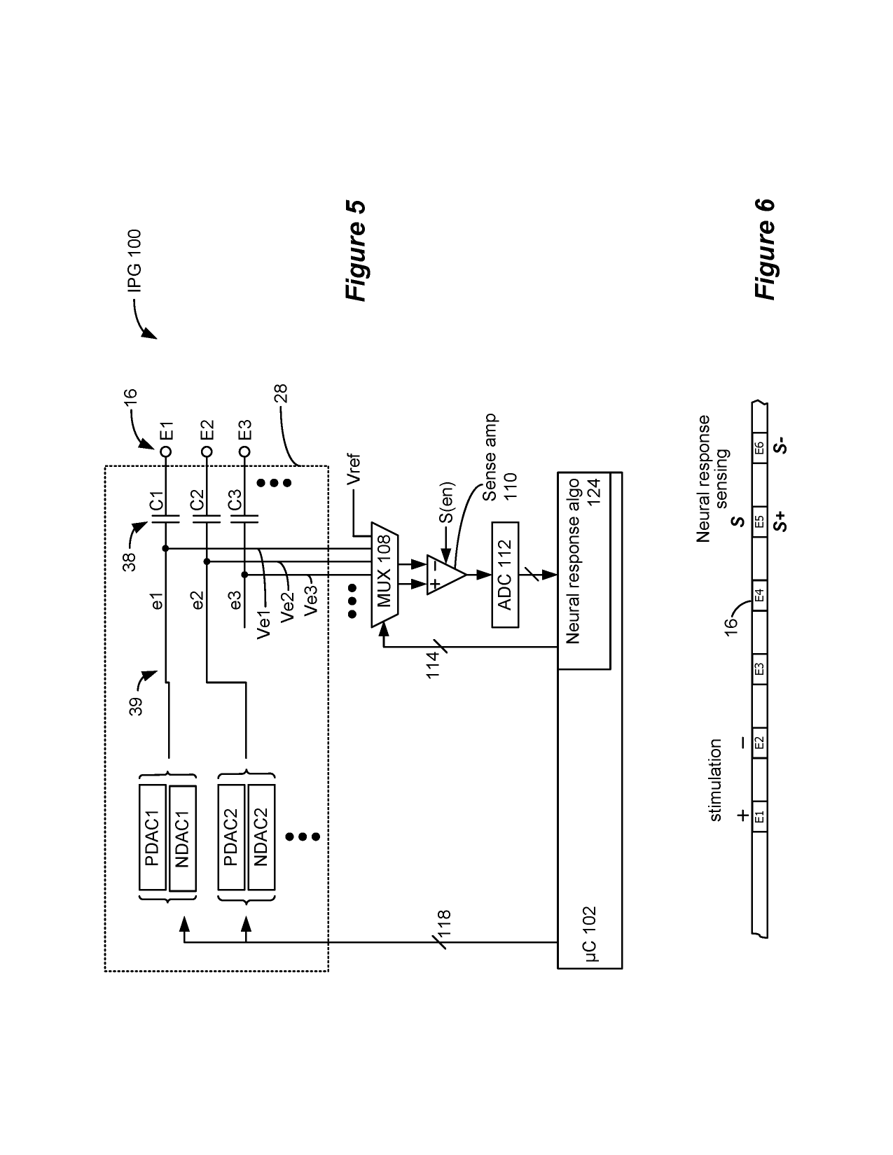

Other stimulation circuitries 28 can also be used in the IPG 10. In an example not shown, a switching matrix can intervene between the one or more PDACs and the electrode nodes ei 39, and between the one or more NDACs and the electrode nodes. Switching matrices allows any PDAC or NDAC to be connected to any of the electrode nodes. Various examples of stimulation circuitries can be found in USPs 6,181,969, 8,606,362, 8,620,436, 11,040,192, and 10,912,942. Much of the stimulation circuitry 28 of FIG. 3, including the PDACs and NDACs, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as IPG master control circuitry 102 (see FIG. 5), telemetry circuitry (for interfacing off chip with telemetry antennas 27a and/or 27b), circuitry for generating the compliance voltage VH (as explained next), various measurement circuits, etc.

Power for the stimulation circuitry 28 is provided by a compliance voltage (VH), as described in further detail in U.S. Patent Application Publications 2013/0289665 and 2018/0071520. The compliance voltage VH may be coupled to the source circuitry (e.g., the PDAC(s)), while ground may be coupled to the sink circuitry (e.g., the NDAC(s)), such that the stimulation circuitry 28 is powered by VH and ground. Other power supply voltages may be used with the PDACs and NDACs, and explained in U.S. Patent Application Publication 2018/0071520, but these aren’t shown in FIG. 3 for simplicity.

As described in USP 11,040,202, which is incorporated herein by reference, the compliance voltage VH can be produced by a VH regulator 49. VH regulator 49 receives the voltage of the battery 14 (Vbat) and boosts this voltage to a higher value required for the compliance voltage VH. VH regulator 49 can comprise an inductor-based boost converter or a capacitor-based charge pump for example. The regulator 49 can vary the value of VH based on measurements taken from the stimulation circuitry 28. As explained in detail in the ‘202 patent, VH measurement circuitry 51 can be used to measure the voltage drops across the active DACs (e.g., PDAC1 (Vp1) and NDAC2 (Vn2) in the example shown in FIG. 3) in the stimulation circuitry 28. Using such measurements allows VH to be established at an energy-efficient level: high enough to form the prescribed current without loading (i.e., without producing less current that prescribed), yet low enough to not needlessly waste power in the stimulation circuitry 28 when forming the prescribed current.

The VH measurement circuitry 51 can output an enable signal VH(en1) indicating when VH regulator 49 should increase the level of VH, i.e., when the voltage drops across the active DACs are too low. This enable signal VH(en1) may be processed at logic 53 in conjunction with other signals explained below to determine a master enable signal VH(en) for the VH regulator 49. Logic 53 may be associated with the IPG’s control circuitry 102. Master enable signal VH(en) when asserted causes the VH regulator 49 to increase VH (e.g., when the current starts to load). Deasserting VH(en) disables the VH regulator, which allows VH to naturally decrease over time until it needs to be increased again. This feedback generally causes VH to be established at an energy-efficient value appropriate for the current that is being provided by the stimulation circuitry 28.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861.

Referring again to FIG. 2A, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38. Charge recovery is shown with reference to both FIGS. 2A and 2B. During the first pulse phase 30a, charge will (primarily) build up across the DC-blockings capacitors C1 and C2 associated with the electrodes E1 and E2 used to produce the current, giving rise to voltages Vc1 and Vc2 (I = C * dV/dt). During the second pulse phase 30b, when the polarity of the current I is reversed at the selected electrodes E1 and E2, the stored charge on capacitors C1 and C2 is recovered, and thus voltages Vc1 and Vc2 hopefully return to 0V at the end the second pulse phase 30b. To recover all charge by the end of the second pulse phase 30b of each pulse (Vc1 = Vc2 = 0V), the first and second phases 30a and 30b are charged balanced at each electrode, with the phases comprising an equal amount of charge but of the opposite polarity. In the example shown, such charge balancing is achieved by using the same pulse width (PWa = PWb) and the same amplitude (|+I| = |-I|) for each of the pulse phases 30a and 30b. However, the pulse phases 30a and 30b may also be charged balance if the product of the amplitude and pulse widths of the two phases 30a and 30b are equal, as is known.

Charge recovery using phases 30a and 30b is said to be “active” because the P/NDACs in stimulation circuitry 28 actively drive a current, in particular during the last phase 30b to recover charge stored after the first phase 30a. However, such active charge recovery may not be perfect, and some residual charge may be present in capacitive structures even after phase 30b is completed. Accordingly, the stimulation circuitry 28 can also provide for passive charge recovery. Passive charge recovery is implemented using passive charge recovery switches PRi 41 as shown in FIG. 3. These switches 41 when selected via assertion of control signals <Xi> couple each electrode node ei to a passive recovery voltage Vpr established on bus 43. As explained in USPs 10,716,937 and 10,792,491, this allows any stored charge to be passively recovered through the patient’s tissue, R, without actively driving currents using the P/NDACs. Control signals <Xi> are usually asserted to cause passive charge recovery after each pulse (e.g., after each last phase 30b) during periods 30c shown in FIG. 2A. Because passive charge recovery involves capacitive discharge through the resistance R of the patient’s tissue, such discharge manifests as an exponential decay in current, as shown in FIG. 2A.

As also discussed in the ‘937 patent, each of the passive charge recovery switches 41 can be associated with a variable resistance, and as such each switch 41 can be controlled by a bus of signals <Xi> to control the resistance at which passive charge recovery occurs—i.e., the on resistance of the switches 41 when they are closed. Note that the common voltage Vpr used during passive charge recovery can comprise ground, VH, VH/2, the voltage of the battery 14 (Vbat), or any other DC voltage provided by the IPG 10, and any number of generator circuits (not shown) can be used to produce these voltages for Vpr. Passive charge recovery during period 30c may be followed by a quiet period 30d during which no active current is driven by the DAC circuitry, and none of the passive recovery switches 41 are closed. This quiet period 30d may last until the next pulse is actively produced (e.g., phase 30a). Like the particulars of pulse phases 30a and 30b, the occurrence of passive charge recovery (30c) and any quiet periods (30d) can be prescribed as part of the stimulation program.

Although not illustrated, the pulses provided by the IPG 10 may also be single-phase (i.e., monophasic) pulses having a single polarity, and thus may lack a second (opposite polarity) pulse phase that provides active charge recovery. Performing passive charge recovery can be more important when monophasic pulses are used, and indeed may be required, as discussed further later.

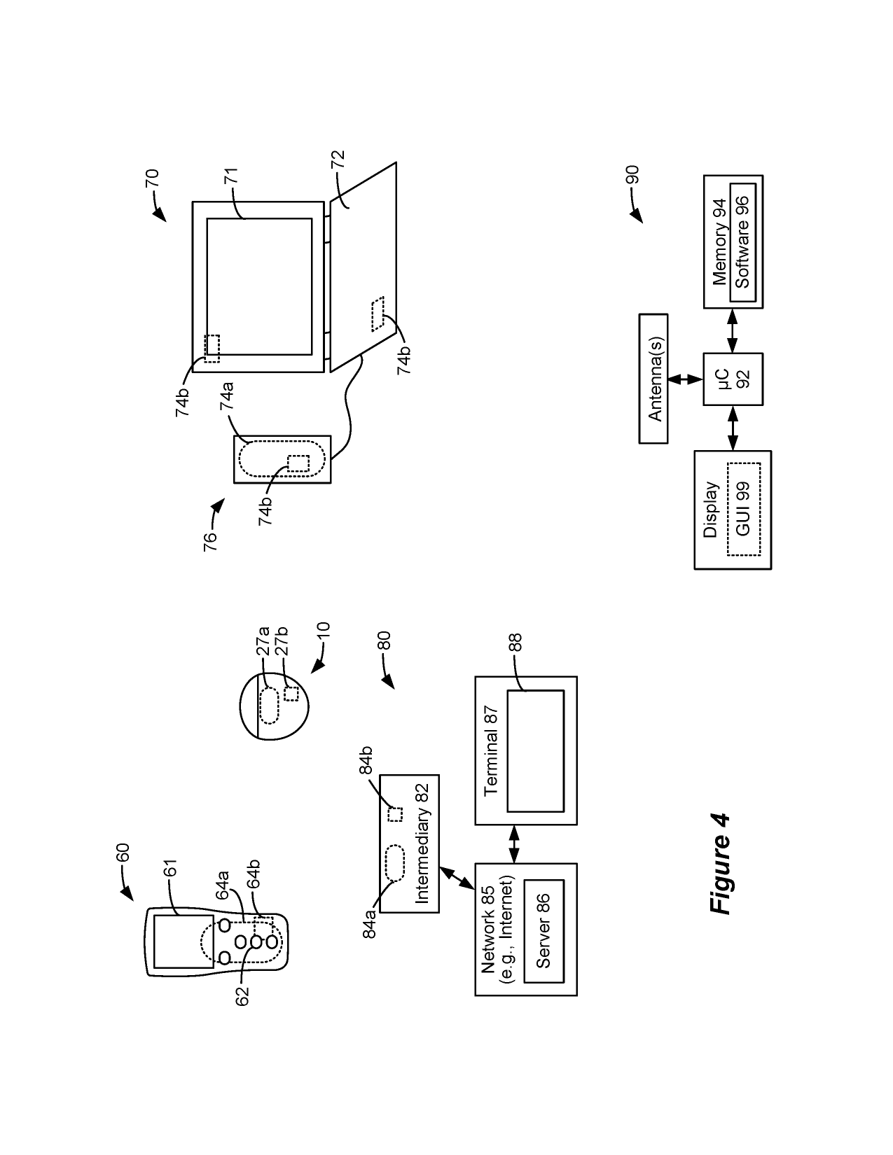

FIG. 4 shows various external systems 60, 70, and 80 that can wirelessly communicate data with the IPG 10 (which again can include an ETS). Such systems can be used to wirelessly transmit a stimulation program to the IPG 10—that is, to program its stimulation circuitry 28 to produce stimulation with desired amplitudes and timings as described earlier. Such systems may also be used to adjust one or more stimulation parameters of a stimulation program that the IPG 10 is currently executing, and/or to wirelessly receive information from the IPG 10, such as various status information, etc.

External controller 60 can be as described in U.S. Patent Application Publication 2015/0080982 for example, and may comprise a portable, hand-held controller dedicated to work with the IPG 10. External controller 60 may also comprise a general-purpose mobile electronics device such as a mobile phone which has been programmed with a Medical Device Application (MDA) allowing it to work as a wireless controller for the IPG 10, as described in U.S. Patent Application Publication 2015/0231402. External controller 60 includes a display 61 and a means for entering commands, such as buttons 62 or selectable graphical icons provided on the display 61. The external controller 60’s user interface enables a patient to adjust stimulation parameters, although it may have limited functionality when compared to systems 70 and 80, described shortly. The external controller 60 can have one or more antennas capable of communicating with the IPG 10. For example, the external controller 60 can have a near-field magnetic-induction coil antenna 64a capable of wirelessly communicating with the coil antenna 27a in the IPG 10. The external controller 60 can also have a far-field RF antenna 64b capable of wirelessly communicating with the RF antenna 27b in the IPG 10.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 4, the computing device is shown as a laptop computer that includes typical computer user interface means such as a display 71, buttons 72, as well as other user-interface devices such as a mouse, a keyboard, speakers, a stylus, a printer, etc., not all of which are shown for convenience. Also shown in FIG. 4 are accessory devices for the clinician programmer 70 that are usually specific to its operation as a stimulation controller, such as a communication “wand” 76 coupleable to suitable ports on the computing device. The antenna used in the clinician programmer 70 to communicate with the IPG 10 can depend on the type of antennas included in the IPG 10. If the patient’s IPG 10 includes a coil antenna 27a, wand 76 can likewise include a coil antenna 74a to establish near-field magnetic-induction communications at small distances. In this instance, the wand 76 may be affixed in close proximity to the patient, such as by placing the wand 76 in a belt or holster wearable by the patient and proximate to the patient’s IPG 10. If the IPG 10 includes an RF antenna 27b, the wand 76, the computing device, or both, can likewise include an RF antenna 74b to establish communication with the IPG 10 at larger distances. The clinician programmer 70 can also communicate with other devices and networks, such as the Internet, either wirelessly or via a wired link provided at an Ethernet or network port.

External system 80 comprises another means of communicating with and controlling the IPG 10 via a network 85 which can include the Internet. The network 85 can include a server 86 programmed with communication and control functionality, and may include other communication networks or links such as WiFi, cellular or land-line phone links, etc. The network 85 ultimately connects to an intermediary device 82 having antennas suitable for communication with the IPG’s antenna, such as a near-field magnetic-induction coil antenna 84a and/or a far-field RF antenna 84b. Intermediary device 82 may be located generally proximate to the IPG 10. Network 85 can be accessed by any user terminal 87, which typically comprises a computer device associated with a display 88. External system 80 allows a remote user at terminal 87 to communicate with and control the IPG 10 via the intermediary device 82.

FIG. 4 also shows circuitry 90 involved in any of external systems 60, 70, or 80. Such circuitry can include control circuitry 92, which can comprise any number of devices such as one or more microprocessors, microcomputers, FPGAs, DSPs, other digital logic structures, etc., which are capable of executing programs in a computing device. Such control circuitry 92 may contain or coupled with memory 94 which can store external system software 96 for controlling and communicating with the IPG 10, and for rendering a Graphical User Interface (GUI) 99 on a display (61, 71, 88) associated with the external system. In external system 80, the external system software 96 would likely reside in the server 86, while the control circuitry 92 could be present in either or both the server 86 or the terminal 87.

An increasingly interesting development in pulse generator systems is the addition of sensing capability to complement the stimulation that such systems provide. For example, and as explained in U.S. Patent Application Publication 2017/0296823, it can be beneficial to sense a neural response produced by neural tissue that has received stimulation from an IPG. U.S. Patent Application Publication 2017/0296823 shows an example where sensing of neural responses is useful in an SCS context, and in particular discusses the sensing of Evoked Compound Action Potentials, or “ECAPs.” U.S. Patent Application Publication 2022/0040486 shows an example where sensing of neural responses is useful in a DBS context, and in particular discusses the sensing of Evoked Resonant Neural Activity, or “ERNA.”

FIG. 5 shows circuitry for sensing neural responses in an IPG 10. The IPG 10 includes control circuitry 102, which may comprise a microcontroller for example, such as Part Number MSP430, manufactured by Texas Instruments, which is described in data sheets accessible on the Internet. Other types of control circuitry may be used in lieu of a microcontroller as well, such as microprocessors, FPGAs, DSPs, or combinations of these, etc. Control circuitry 102 may also be formed in whole or in part in one or more Application Specific Integrated Circuits (ASICs) in the IPG 10 as described earlier, which ASIC(s) may additionally include the other circuitry shown in FIG. 5.

FIG. 5 includes the stimulation circuitry 28 described earlier (FIG. 3), including one or more DACs (PDACs and NDACs). A bus 118 provides digital control signals to the DACs to produce currents or voltages of prescribed amplitudes and with the correct timing at the electrodes selected for stimulation. The electrode current paths to the electrodes 16 include the DC-blocking capacitors 38 described earlier.

FIG. 5 also shows circuitry used to detect neural responses. As shown, the electrode nodes 39 are input to a multiplexer (MUX) 108. The MUX 108 is controlled by a bus 114, which operates to select one or more electrode nodes, and hence to designate corresponding electrodes 16 as sensing electrodes. The sensing electrode(s) selected via bus 114 can be determined automatically by control circuitry 102 and/or a neural response algorithm 124, as described further below. However, the sensing electrode(s) may also be selected by the user (e.g., a clinician) via an external system (FIG. 4).

Electrodes selected as sensing electrodes are provided by the MUX 108 to neural response detection circuitry. This circuitry can comprise a sense amplifier 110, and sensing can occur differentially using two sensing electrodes, or using a single sensing electrode. This is shown in the example of FIG. 6. If single-ended sensing is used, a single electrode (e.g., E5) is selected as a sensing electrode (S) and is provided to the positive terminal of the sense amp 110, where it is compared to a reference voltage Vref provided to the negative input. The reference voltage Vref can comprise any DC voltage produced within the IPG, such as ground. If differential sensing is used, two electrodes (e.g., E5 and E6) are selected as sensing electrodes (S+ and S-) by the MUX 108, with one electrode (e.g., E5) provided to the positive terminal of the sense amp 110, and the other (e.g., E6) provided to the negative terminal. Differential sensing can be useful to cancel any common mode voltages present in the tissue and reflected at the electrodes, such as voltages created by the stimulation itself. See, e.g., U.S. Patent Application Publication 2021/0236829. Although only one sense amp 110 is shown in FIG. 5 for simplicity, there could be more than one, such as a sense amp dedicated to each electrode node. In this case, MUX 108 would not be necessary, and each sense amp could be activated as needed depending on which electrodes are selected as sensing electrodes. The timing at which sensing occurs can be affected by a sensing enable signal S(en), as discussed further below.

The analog waveform comprising the sensed neural response and output by the sense amp 110 is preferably converted to digital signals by an Analog-to-Digital converter (ADC) 112, and input to the IPG’s control circuitry 102. The ADC 112 can be included within the control circuitry 102’s input stage as well. The control circuitry 102 can be programmed with a neural response algorithm 124 to evaluate the neural response, and to take appropriate actions as a result. For example, the neural response algorithm 124 may change the stimulation in accordance with the sensed neural response and can issue new control signals via bus 118 to change operation of the stimulation circuitry 28 to affect better treatment for the patient. The neural response algorithm 124 may also cause the selection of new sensing electrode(s), which can be affected by issuing new control signals on bus 114. Selecting optimal sensing electrode(s) can be important and may be determined in light of stimulation that is being provided. In this regard, sensing electrodes may be selected near enough to the electrodes providing stimulation (e.g., E1 and E2) to allow for proper neural response sensing, but far enough from the stimulation that the stimulation doesn’t substantially interfere with neural response sensing. See, e.g., U.S. Patent Application Publication 2020/0155019.

Neural responses to stimulation are typically small-amplitude signals on the order of microVolts or milliVolts, which can make sensing difficult. The sense amp 110 needs to be capable of resolving this small signal, and this is particularly difficult when one realizes that this small signal typically rides on a background voltage otherwise present in the tissue. As explained in USP 11,040,202, which is incorporated by reference in its entirety, this background voltage can vary on the order of Volts, and can be caused by the stimulation itself. It is difficult to design sense amplifier circuitry 110 to reliably perform the task of accurately sensing a small-signal neural response while rejecting the background tissue voltage. Because stimulation causes the background tissue voltage to vary, it is preferred that neural responses are sensed after active stimulation is provided. Thus, sensing enable signal S(en) is preferably asserted during these times. That being said, stimulation artifacts resulting from the stimulation may still be present and cause variations in the tissue voltage even after stimulation has ceased. See, e.g., PCT (Int’l) Patent Application Publication WO 2020/251899.

One way of addressing this issue of background tissue voltage variability is to drive and hold the tissue to a pseudo-constant common mode voltage (Vcm). The above-incorporated ‘202 patent (585-0281US) and U.S. Patent Publication No. 2023/0138443 (585-0269US), which is incorporated herein by reference, each describe circuitry to hold the tissue to a pseudo-constant programmable common mode voltage (Vcm). In these incorporated references, the primary purpose of biasing the tissue at a common mode voltage Vcm is to facilitate the ability to sense neural responses and other potentials within the patient’s tissue. The inventors have realized that providing such common mode voltages can also be used for other purposes, such as preventing malfunction of the IMD when a patient is subjected to transient and/or gradient electromagnetic fields, such as during magnetic resonance imaging (MRI). Such MRI protection will be described in more detail below.

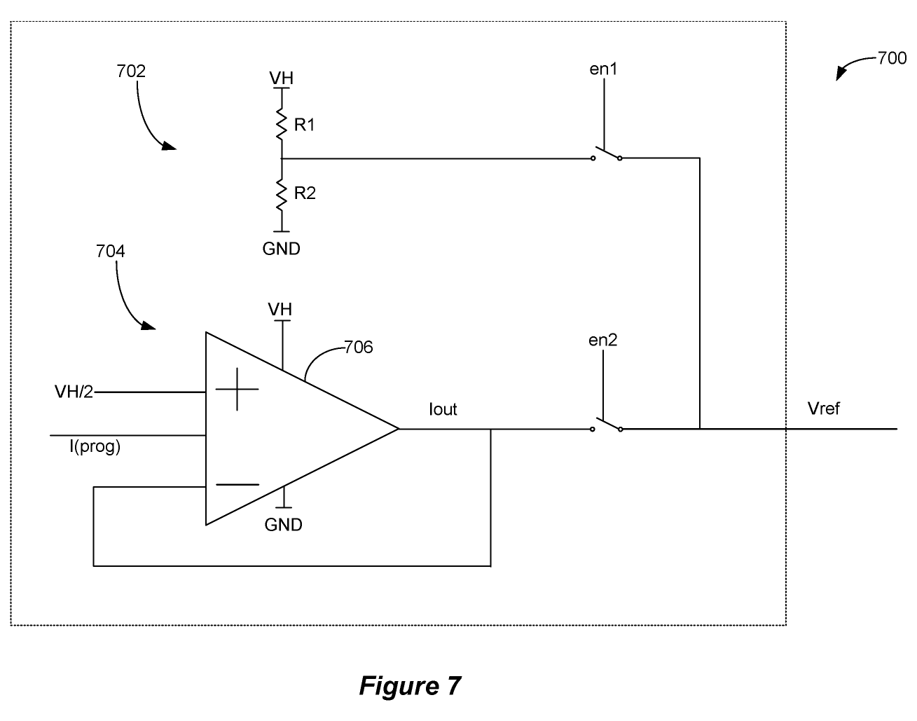

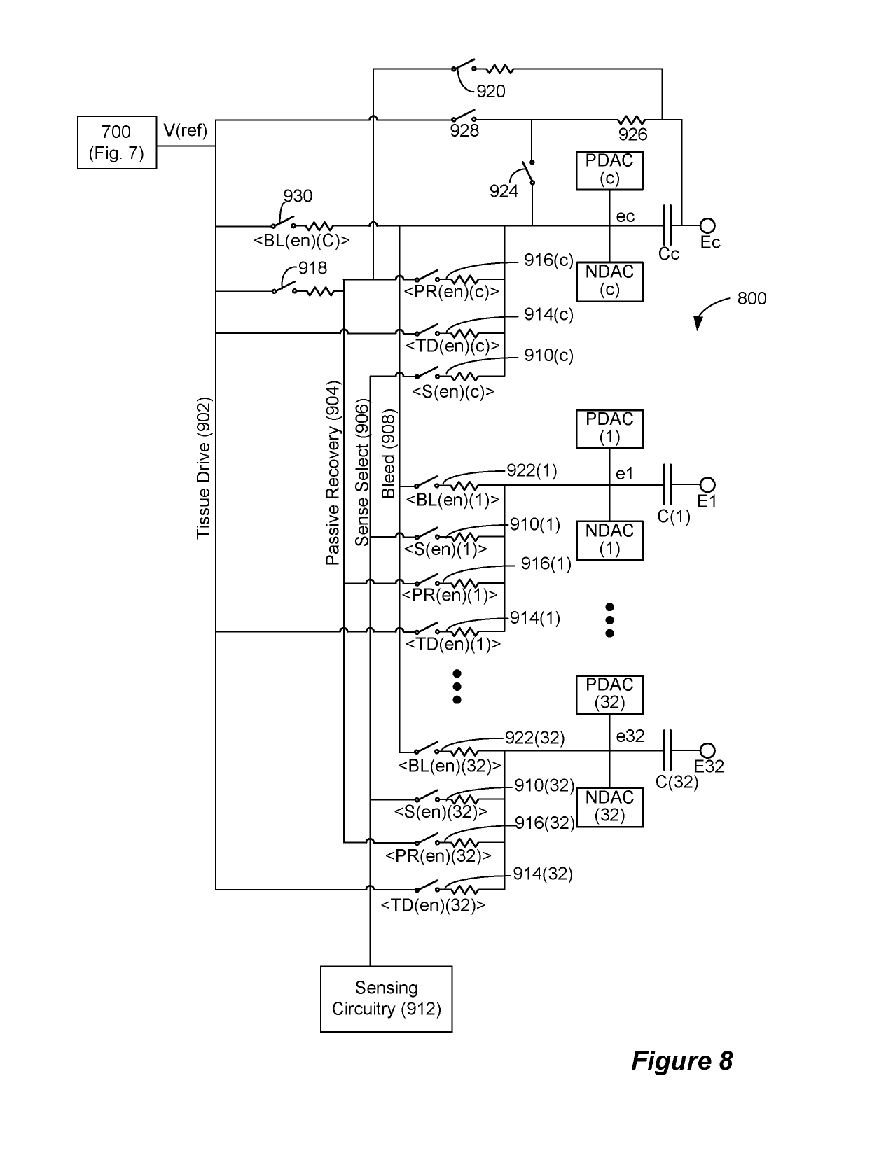

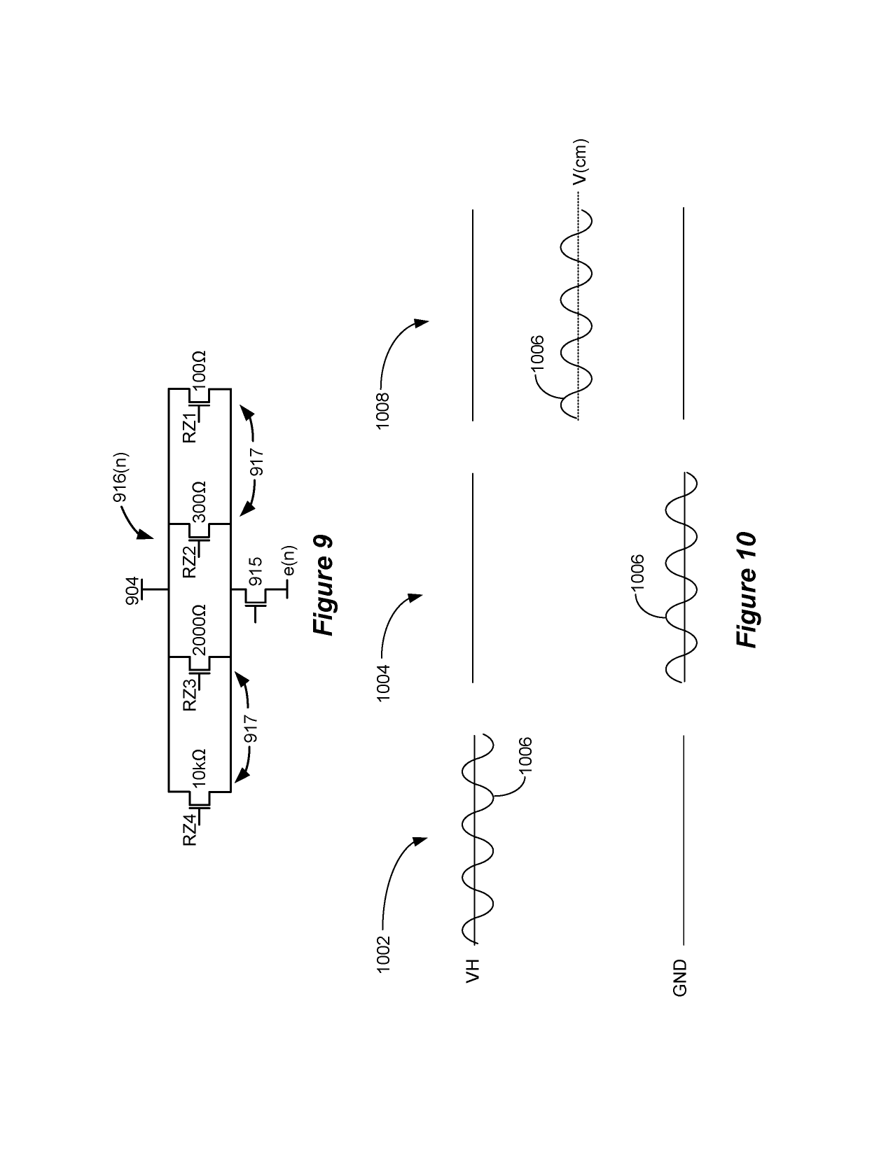

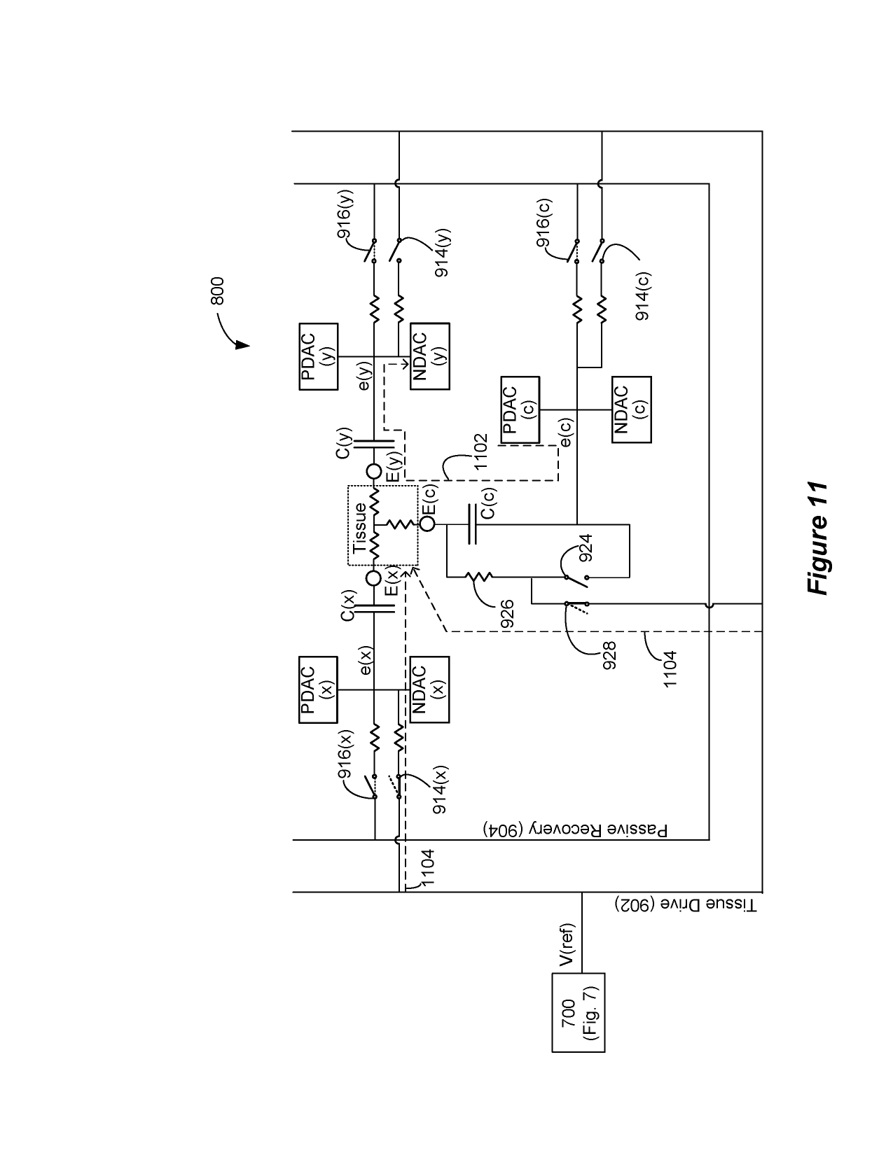

Generally, any circuitry may be used to provide a known common mode voltage (Vcm). FIG. 7 illustrates an example of reference voltage circuitry 700 (also referred to herein as “biasing circuitry”) for providing a reference voltage V(ref) that may be used to drive the tissue to a common mode voltage V(cm). The illustrated circuitry is configured to provide a reference voltage of VH/2, but it is within the ability of a person of skill in the art to configure similar circuitry for providing other reference voltage values. The illustrated circuitry comprises two selectable driver circuits 702 and 704 for driving Vref. The either 702 or 704 may be selected by asserting enable signals en1 or en2, respectively. The driver circuit 702 is referred to herein as high impedance driver circuitry. The high impedance circuit 702 comprises a voltage divider comprising resistors R1 and R2 to divide the compliance voltage VH. R1 and R2 may be equal to provide a reference voltage of VH/2. R1 and R2 may each be 10 MΩ, for example.

The driver circuit 704 is referred to herein as a programmable driver circuitry. Such programmable driver circuitry is described in more detail in the above-incorporated ‘202 patent. The programmable driver circuitry 704 comprises an amplifier 706 that may be configured as a voltage follower that is configured to drive a load to maintain the reference voltage provided at the positive input of the amplifier (VH/2 in the illustrated embodiment). As described in the ‘202 patent, the amplifier may operate as an operational transconductance amplifier (OTA). The maximum output current Iout (i.e., the drive strength) of the amplifier 706 may be controlled using a programmable input I(prog). Accordingly, a buffer associated with the amplifier 704 may receive control signals to program the drive strength of programmable driver circuitry 704. Such programmable drive strength may be beneficial when biasing the tissue. For example, when the case electrode, which has a high surface area, is used as a biasing electrode, limiting the drive strength may be used to prevent "pocket stimulation," which is when the case stimulates the tissue pocket in which the case is implanted. In some embodiments, the drive strength may be incrementally programmed to deliver currents of about 150 µA to about 5 mA.

FIG. 8 illustrates aspects of IPG circuitry 800 for providing stimulation and sensing of tissue electrical signals. Aspects of the IPG circuitry are described in more detail in U.S. Provisional Application No. 63/705,630, filed October 10, 2024, entitled “Implantable Stimulator Device with Controllable Bleed Resistors at all Electrodes,” the entire contents of which are hereby incorporated by reference. The illustrated IPG circuitry is configured for 32 lead-based electrodes and a case electrode. The electrodes are denoted as Ec (i.e., case electrode) and E1-E32. Each electrode is associated with PDACs and NDACs, electrode nodes ec-e32, and blocking capacitors C(c)-C32, which are as described above.

Four buses are illustrated in the embodiment shown in FIG. 8. The buses are introduced here, and specific implementations of the buses are described in more detail below. Bus 902 is referred to as a tissue drive bus. The bus 902 is connected to a reference voltage (V(ref)) source. The source of V(ref) may be a circuit, such as circuit 700 (FIG. 7). One of the uses of the bus 902 is to drive the patient’s tissue to a common mode reference voltage. Bus 904 is referred to as the passive recovery bus and is implemented in passive charge recovery, which is described above. Bus 906 is referred to as a sense select bus and is used to instantiate given electrodes for sensing/recording. Bus 908 is referred to as a bleed bus and is used to provide a DC path to bleed accumulated residual charge on the blocking capacitors.

As with IPG circuitry discussed above (FIG. 3), the PDACs/NDACs of any of the electrode nodes ec – e32 of the IPG circuitry 900 may be digitally programmed to provide current to their respective electrodes. Control and power circuitry for the PDACs/NDACs is omitted from FIG. 8 for the sake of clarity, but the reader is referred to FIG. 3, for example.

Any of the electrode nodes/electrodes of the IPG circuitry 800 may also be enabled for sensing/recording electrical potentials present in the patient’s tissue. In the illustrated embodiment, sense select switches 910(c) – 910(32) are associated with each of the electrode nodes ec – e32, respectively. The sense select switches may be enabled by a sense enable signal <S(en)(n)> to connect the respective electrode node to sensing circuitry 912 via the sense select bus 906. According to some embodiments, the sensing circuitry 912 may comprise a plurality of sense amplifiers. For example, U.S. Patent Application Publication 2025/0269185, the entire contents of which are incorporated herein by reference, describes sensing circuitry including a downselector with switching networks used to couple selected electrode nodes and/or DC voltages to one or more selected sense amplifiers. The downselector can be controlled to couple one or more electrodes nodes to a particular sense amp during sensing durations. According to some embodiments, the enablement signal <S(en)(n)> may comprise multiple bits of information configured to control the switching network/downselector.

As mentioned above, the embodiments of the IPG circuitry 800 are configured to drive the tissue to a common mode reference voltage V(ref), e.g., VH/2, VH, Vbat, ground, or the like. Accordingly, each electrode node ec – e32 is configured with a tissue drive switch 914(c) – 914(32), respectively. Each of the respective tissue drive switches may be enabled by an tissue drive enable signal <TD(n)> that may be asserted to connect the respective electrode node e(n) to the tissue drive bus 902.

Each electrode node ec – e32 is also configured with a passive charge recovery switch 916(c) – 916(32) that may be enabled to effect passive charge recovery, as described above. Each of the passive charge recovery switches may be enabled by a passive charge recovery enable signal <PR(en)(n)>, which may be asserted to connect the respective electrode node to the passive recovery bus 904. According to some embodiments, each of the passive charge recovery switches may comprise variable resistors (or a plurality of switches) configured to provide a variable resistance for passive charge recovery. For example, U.S. Patent No. 10,716,937 (“the ‘937 Patent”), the entire contents of which are incorporated herein by reference, describes such a network for providing variable and programmable resistance during passive charge recovery. FIG. 9 illustrates an embodiment of such a passive charge recovery switch 916(n), as described in the ‘937 Patent, connected between an electrode node e(n) and the passive charge recovery bus 904. The passive charge recovery switch 916(n) comprises a first switch 915 serially connected to a network of resistance transistor/switches 917 that are in parallel with each other. Resistance control signals RZ[4:1] are each received at the gate of one of the resistance transistor/switches 917. Each of resistance transistors 917 is preferably sized differently to provide a different resistance. In the example shown, RZ1 controls a resistance transistor 917 of 100 ohms; RZ2 controls 300 ohms; RX3 controls 2000 ohms; and RZ4 controls 10000 ohms. Accordingly, the passive charge recovery enable signal <PR(en)(n)> may comprise the bits of information configured to select the resistance of the respective passive charge recovery switch 916(n). It should be noted that the resistance transistor/switches 917 are referred to herein as “resistors” even though they are implemented as transistor/switches in some embodiments.

The illustrated IPG circuitry 800 provides two modes for passive charge recovery. In a first mode, charge stored on the blocking capacitors C(n) can be recovered during passive charge recovery by connecting the passive charge recovery bus 904 to the tissue drive bus 902 via the switch 918. The switch 918 may be associated with a resistor, as shown, which may have a large value, for example, about 5 MΩ. Thus, the charge on the blocking capacitors C(n) may be recovered to V(ref). In a second mode, the charge on the blocking capacitors C(n) may be provided to the case electrode Ec via a switch 920, where the charge may be dissipated through the tissue impedance. The switch 920 may also be associated with a resistor, which according to some embodiments, may be a variable resistor. This is particularly useful for removing DC bias on the case blocking capacitor C(c). In the rest of this disclosure, the two various modes or passive charge recovery will not be distinguished.

In some instances, passive charge recovery may not completely discharge accumulated charge on the blocking capacitors. Thus, the embodiments of the IPG circuitry 800 include various bleed resistors and DC paths configured to bleed residual charge off the blocking capacitors. The electrode nodes e1 – e32 for each of the lead-based electrodes are connected to bleed resistors/switches 922(1) – 922(32), respectively. The bleed resistors typically have a high resistance, for example, about 5 MΩ. The bleed resistors/switches may each be individually controlled via a bleed enable signal <BL(en)(n)>, which when asserted, connect the respective electrode node to the bleed bus 908 through the bleed resistor. Notably, the bleed resistors for each of the electrode nodes may be individually selected and controlled. The residual charge that is bled to the bleed bus 908 may be bled to the case electrode by closing a switch 924. That DC path includes a resistor 926, which may be about 1 MΩ, for example. That DC path may be driven to V(ref) by closing a switch 928. Each of these switches may be controlled using appropriate control signals, which are omitted in the drawing for the sake of clarity. Note that when the switches 924 and 928 are both open, the bleed bus 908 simply bleeds the residual charge from the lead-based electrode nodes e1 – e32 to the inside of the case electrode’s blocking capacitor Cc. The case electrode node ec is also equipped with a bleed resistor/switch 930, which may also have a resistance of about 5 MΩ. The case bleed electrode may be controlled by a case bleed enable signal <BL(en)(c)>. Closing the case bleed resistor/switch 930 bleeds charge stored on Cc to the reference voltage V(ref). Likewise, closing switch 924 bleeds charge stored on Cc to the case electrode itself through the resistor 926 and to the tissue resistance.

It will be apparent to a person of skill in the various busses and programmable switches of the IPG circuitry 800 illustrated in FIG. 8 provides substantial flexibility in signals and voltages may be managed within the IPG and with respect to the various electrodes. This disclosure is primarily concerned with using circuitry within the IPG to bias a patient’s tissue at a common mode voltage Vcm when the patient is undergoing MRI. In some embodiments, the disclosed IPGs may be configured with an “MRI-safe mode,” whereby actions are taken to protect the IPG and to protect the patient from unintended stimulation.

It is known in the art that when a patient undergoes MRI their body is exposed to varying electromagnetic fields and field gradients. Such fields and field gradients can induce voltages within the patient’s tissue and within portions of an implantable medical device. Even if stimulation is not provided to the patient during the MRI procedure, the induced voltages can cause malfunctions within the implantable device and may, in some instances, result in the device providing unwanted stimulation. Referring to FIG. 10, scenarios 1002 and 1004 illustrate examples of allowing the patient’s tissue voltage 1006 to “float” under the influence of the external electromagnetic fields. Note that the tissue voltage 1006 may include an AC component, as illustrated. If some or all of the tissue voltage signal exceeds the compliance voltage VH (as shown in scenario 1002) or falls below the ground voltage (as shown in scenario 1004), the tissue voltage may turn on diodes of the IPG’s ASIC circuitry, which may create a path for current to flow into the patient’s tissue (i.e., unintended, and potentially harmful stimulation).

The inventors have recognized that the tissue driving circuitry, such as described above, may be leveraged to address the problems associated with induced tissue voltages during MRI. As shown in scenario 1008, the tissue driving circuitry may be configured to bias the patient’s tissue at a common mode voltage Vcm, such that the tissue voltage does not exceed VH or drop below GND. In the illustrated example, the tissue voltage is biased mid-rail, that is, at VH/2. As mentioned above, other Vcm values may be chosen. But VH/2 is preferable in some embodiments so as to minimize the likelihood of AC components of the induced tissue voltages from going above VH or below GND.

Embodiments of the disclosed IPGs and medical device systems may feature an MRI-safe mode, which when instantiated, may take actions to ready the IPG for an MRI procedure. In some embodiments, the MRI-safe mode may be instantiated in response to instructions received via the patient’s external controller 60 or another external device. In other embodiments, the MRI-safe mode may be instantiated in response to indications that the patient is undergoing an MRI procedure. For example, the IPG may be equipped with a magnetic field detector (e.g., Hall effect sensor) or other detector, such as an accelerometer configured to detect indicia of an MRI procedure.

In some embodiments, the MRI-safe mode may be configured to increase the compliance voltage VH. For example, in embodiments configured with programmable compliance voltage, as described in the incorporated ‘202 patent, entering the MRI-safe mode may send a control signal to automatically increase the compliance voltage.

In some embodiments, therapeutic stimulation is ceased during the MRI-safe mode and the patient does not receive stimulation while they are undergoing the MRI procedure. In other embodiments, stimulation can be provided in the MRI-safe mode.

In some embodiments, entering the MRI-safe mode configures the IPG to assert a biasing common mode voltage Vcm to the patient’s tissue, as described above. As a first example, consider scenarios in which no therapeutic stimulation (or sensing) occurs during the MRI-safe mode. In a first step, the reference voltage circuitry 700 (FIG. 7) is configured to generate a common mode voltage Vcm (e.g., VH/2) to bias the patient’s tissue. This may involve selecting either the high impedance driver circuitry 702 (by asserting the control signal en1) or the programmable driver circuitry 704 (by asserting the control signal en2). If the programmable driver circuitry 704 is selected, a control signal to program the programmable bias current I(prog) may be provided to the buffer of the amplifier 706 to regulate the drive current Iout provided by the driver circuitry. In some embodiments, the selection of the driver circuitry and the I(prog) (if appropriate) may be preprogrammed as default selections to be executed when entering the MRI-safe mode. In some embodiments, those selections may be entered via the patient’s external controller or another external device. I some embodiments, the IPG may be programmed with a plurality of MRI-safe modes, each with different selections of those values, and the user may select a particular MRI-safe mode.

As a further step, one or more of the electrode nodes are selected to provide the common mode voltage to the tissue. As mentioned above, in some embodiments the case electrode node may be preferable for this purpose, owing to the relatively large surface area of the case electrode. But generally, any electrode (or electrodes) may be selected, given the flexibility of the IPG circuitry 800 (FIG. 8). For this example, assume that the case electrode is selected to bias the tissue during the MRI-safe mode. Referring to FIG. 8, the case electrode may be configured to provide the common mode voltage by closing the switch 914(c) (i.e., by asserting the enable signal <TD(en)(c)>) or by closing switch 928, for example. Other electrode nodes may be selected to provide the common mode voltage by closing their respective tissue drive switches 914(n).

The IPG circuitry 800 may also be configured to deliver stimulation when the IPG is in an MRI-safe mode. For example, the case and/or one or more lead-based electrode nodes may be configured to provide common mode voltage to the tissue while stimulation is delivered. In such embodiments, the tissue drive switches of one or more electrode node (e.g., the case electrode node) may be closed to bias the patient’s tissue at a common mode voltage while the PDACs/NDACs and passive recovery switches of selected electrode nodes are employed to provide stimulation and passive charge recovery.

FIG. 11 shows an instantiation using IPG circuitry 800 to deliver a phase of monopolar stimulation 1102 to a patient’s tissue (which is illustrated as a resistance in the illustration) by implementing the case PDAC(c) to source current and the NDAC(y) of a lead-base electrode node e(y) to sink current. Note that only the tissue drive rail 902 and the passive charge recovery rail 904 and their associated switches are shown in FIG. 11. That is, the sense select and bleed rails are omitted for clarity (compare to FIG. 8). In the MRI-safe mode in the illustrated embodiment, one or more lead-based electrode nodes, such as the electrode node e(x) and/or the case electrode node e(c) may be used to provide current 1104 to bias the tissue at a common mode voltage Vcm, for example, by closing switches 914(x) and 928 (or 914(c)), respectively. As explained above, the tissue biasing current may be provided using either the high impedance driver circuitry or the programmable driver circuitry of the reference voltage circuitry 700. During the passive charge recovery phases of the stimulation waveform the switches 916(x), 916(y), and 916(c) may be closed to effect passive charge recovery, as explained above. Including the referencing electrodes during the passive charge recovery phase(s) promotes rebalancing charges at all electrode nodes used during the process.

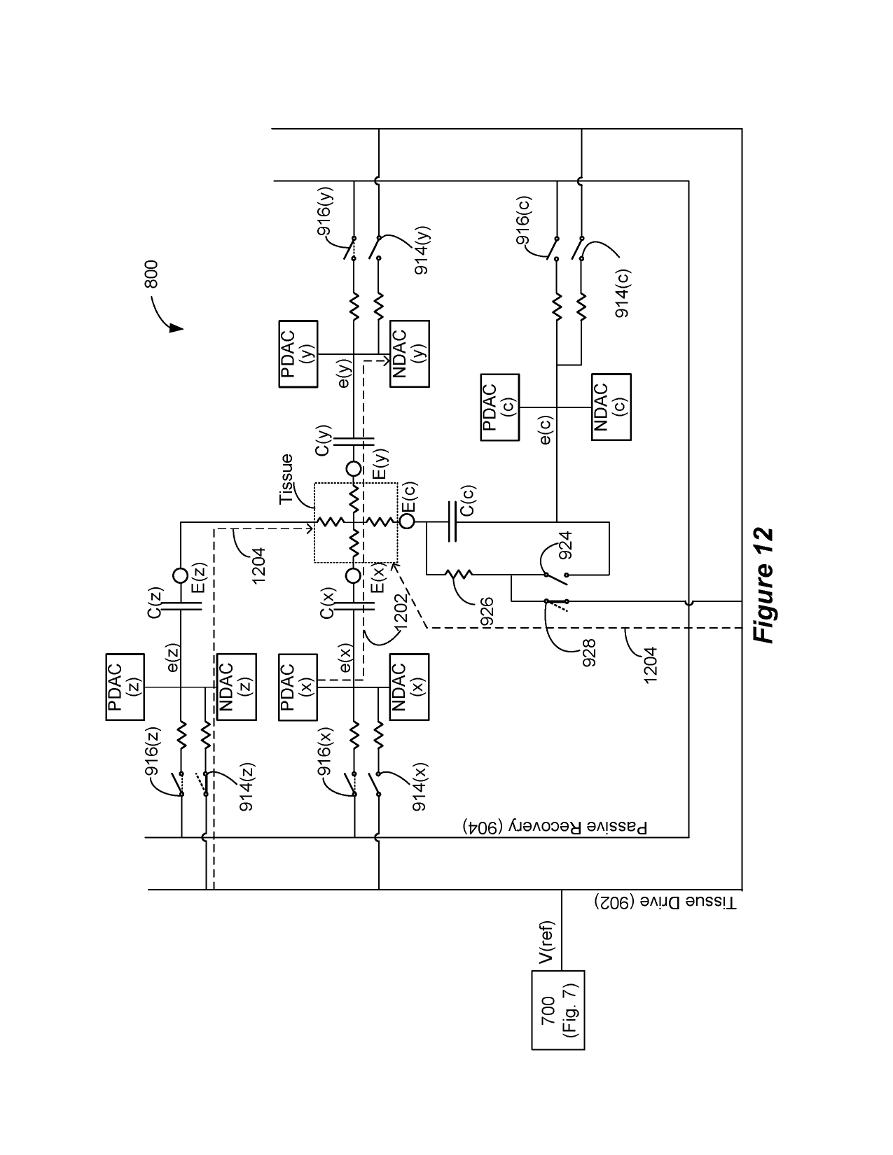

FIG. 12 shows an instantiation using IPG circuitry 800 to deliver a phase of bipolar stimulation 1202 to a patient’s tissue during an MRI-safe mode. The PDAC(x) of a first lead-based electrode node e(x) is used to source the current 1202 and the NDAC(y) of a second lead-base electrode node e(y) sinks the current. In the illustrated embodiment, a lead-based electrode node e(z) and/or the case electrode node e(c) may be configured to provide current 1204 to bias the tissue at a common mode voltage Vcm, for example, by closing switches 914(z) and/or 928, respectively. During the passive charge recovery phases of the stimulation waveform the switches 916(x) and 916(y) may be closed to effect passive charge recovery, as explained above.

It will be apparent to a person of skill in the art that the flexibility and programming provided by circuitry 800 (FIG. 8) provides flexibility in effecting controlled tissue biasing during stimulation, particularly while in the MRI-safe mode. Other timings and schemes will be apparent to those of skill in the art.

Although particular embodiments of the present invention have been shown and described, the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. An implantable stimulator device configured to provide electrical stimulation to a patient, comprising:
a plurality of electrode nodes each couplable to a different electrode configured to contact the patient’s tissue,
stimulation circuitry configured to drive at least two of the electrode nodes to provide current through the tissue,
biasing circuitry configured to produce a reference voltage,
a plurality of first switches each associated with a different one of the electrode nodes, wherein each of the plurality of first switches is configured, when selected, to couple the reference voltage to its associated electrode node to provide a common mode voltage to the patient’s tissue at that node’s associated electrode, and
control circuitry configured to:
receive a command to put the stimulator device in an MRI-safe mode, and
in response to the command, closing a selected one or more of the first plurality of first switches.

2. The device of claim 1, wherein the selected one or more of the first plurality of switches comprises a first switch associated with a case electrode.

3. The device of claim 1, wherein the selected one or more of the first plurality of switches comprises a first switch associated with an electrode comprised upon an electrode lead that is implantable in the patient’s tissue.

4. The device of claim 1, wherein the stimulation circuitry is configured to be powered between a compliance voltage (VH) and a ground, and wherein the reference voltage is between VH and ground.

5. The device of claim 4, wherein the reference voltage is VH/2.

6. The device of claim 4, wherein the control circuitry is configured to increase VH upon receipt of the command to put the stimulator device in an MRI-safe mode.

7. The device of claim 1, wherein the biasing circuitry comprises a high impedance driver circuitry and a programmable driver circuitry.

8. The device of claim 7, wherein the high impedance driver circuitry comprises a voltage divider.

9. The device of claim 7, wherein the programmable driver circuitry comprises an amplifier configured to produce the reference voltage.

10. The device of claim 9, wherein the amplifier is configured to receive a programmable input bias current that may be programmably selected to control a drive strength of the reference voltage.

11. The device of claim 7, wherein the control circuitry is configured to select between the high impedance driver circuitry and the programmable driver circuitry in the MRI-safe mode.

12. The device of claim 1, wherein the control circuitry is configured to cause the device to cease providing stimulation when in the MRI-safe mode.

13. The device of claim 1, wherein the device is configured to provide stimulation at selected one or more of the electrode nodes when the device is in the MRI-safe mode.

14. The device of claim 13, wherein the stimulation comprises at least one active stimulation duration during which the stimulation circuitry actively drives current at at least two selected stimulation electrode nodes, and wherein the control circuitry is configured to:
select the at least two stimulation electrode nodes, and
select one or more biasing electrode nodes for providing the common mode voltage to the patient’s tissue.

15. The device of claim 14, wherein the control circuitry is configured to, in response to the command to put the stimulator device in an MRI-safe mode close the one or more of the plurality of first switches for the biasing electrode nodes.

16. The device of claim 15, wherein the control circuitry is configured to cause the stimulation circuitry to drive the at least two stimulation electrode nodes during the stimulation duration.

17. The device of claim 15, wherein the control circuitry is configured to select between the high impedance driver circuitry and the programmable driver circuitry in response to the command to put the MRI-safe mode.

18. The device of claim 14, wherein the stimulation comprises at least one passive charge recovery duration during which the stimulation does not drive the at least two stimulation electrode nodes, and wherein the control circuitry is configured to close second switches coupled to the at least two stimulation electrode nodes during the passive charge recovery duration to drain charge from DC-blocking capacitors coupled to the at least two stimulation electrode nodes.

19. The device of claim 18, wherein the control circuitry is configured to close second switches coupled to the one or more biasing electrode nodes during the passive charge recovery duration to drain charge from DC-blocking capacitors coupled to the biasing electrode nodes.

20. The device of claim 1, wherein the command to put the stimulator device in an MRI-safe mode is responsive to a sensed magnetic field.
